(19) United States
(12) Patent Application Publication
(10) Pub. No.: US 2009/0275505 A1
(43) Pub. Date: Nov. 5, 2009

(54) Compositions and Methods For Preventing or Treating Feline Chronic Renal Failure

(75) Inventor: Karen Joy Wedekind, Saint Peters, MO (US)

Correspondence Address: COLGATE-PALMOLIVE COMPANY, 909 RIVER ROAD, PISCATAWAY, NJ, 08855, US

(73) Assignee: Hill's Pet Nutrition, Inc., Topeka, KS (US)

(21) Appl. No.: 12/161,583

(22) PCT Filed: Jan. 19, 2007
(86) PCT No.: PCT/US07/60773

§ 371 Date: Jun. 30, 2009

Related U.S. Application Data
(60) Provisional application No. 60/760,068, filed on Jan. 19, 2006.

(51) Int. Cl. A61K 38/16

(52) U.S. Cl. 514/12

(57) ABSTRACT

The invention provides compositions for preventing chronic feline renal failure comprising from about 28 to about 35% protein and compositions for treating chronic renal failure in felines comprising from about 28 to about 30%, wherein the protein comprises at least about 75% vegetable protein and methods for using such compositions to prevent or treat chronic feline renal failure. The invention also provides methods for preparing the compositions, articles of manufacture comprising the compositions, and means for communicating information about the compositions, methods, and articles of manufacture.

### BACKGROUND OF THE INVENTION

1. Field of the Invention

The invention relates generally to compositions for preventing or treating chronic renal failure and particularly to compositions for preventing or treating feline chronic renal failure.

2. Description of the Related Art

The kidneys have five primary functions. They filter waste products from the body (e.g., urea and creatinine), regulate electrolytes (e.g. potassium, calcium, phosphorus, and sodium), produce erythropoietin (which stimulates bone marrow to produce red blood cells), produce rennin (which controls blood pressure), and produce and concentrate urine.

Chronic renal failure (CRF) is a progressive kidney disease which has four phases: loss of renal reserve, renal insufficiency, azotemia, and uremia. The kidneys have a large built-in reserve as only approximately 30% of kidney capacity is needed for normal kidney function. Kidney capacity diminishes with time for a variety of reasons, for example, advanced age and diseases and medications that damage the kidney(s). Renal insufficiency is characterized by decrease in renal function, and is generally observed when about 70% of kidney function has been lost (i.e., when only about 30% of kidney capacity is available). Clinical signs are typically not obvious during the phases of loss of renal reserve and renal insufficiency, thereby making it difficult to detect CRF. Azotemia, the third phase of CRF, is characterized by an abnormally high concentration of urea, creatinine, and/or other non-protein nitrogen-containing substances in the blood. Renal function is markedly reduced in the azotemic phase, although clinical signs may be mild and can go undetected. Uremia is characterized by the presence of abnormal quantities of urine or urine constituents in blood. The uremic phase is characterized by overt clinical signs.

CRF is a terminal disease. It is one of the leading causes of death in felines. Thus, there is a need for compositions and methods for preventing CRF in felines. There is also a need for compositions and methods for treating CRF which provide partial or complete relief.

### SUMMARY OF THE INVENTION

It is, therefore, an object of the invention to provide compositions suitable for preventing CRF in felines.

It is another object to provide compositions suitable for treating CRF in felines.

It is a further object to provide methods for preventing CRF in felines.

It is another object to provide methods for treating CRF in felines.

It is a further object to provide articles of manufacture comprising a composition of the invention or two or more ingredients that, when combined together and, optionally, with additional ingredients, yield a composition of the invention for preventing CRF in a feline.

It is another object to provide articles of manufacture comprising a composition of the invention or two or more ingredients that, when combined together and, optionally, with additional ingredients, yield a composition of the invention for treating CRF in a feline.

It is another object to provide means for communicating information about the compositions, methods, and articles of manufacture of the invention.

One or more of these and other objects are achieved by preventing chronic feline renal failure by feeding the feline a composition comprising from about 28 to about 35% protein on a dry matter basis and treating chronic feline renal failure by feeding the feline a composition that comprises from about 28 to about 30% protein on a dry matter basis, wherein the protein comprises at least about 75% vegetable protein.

Additional objects, features, and advantages of the invention will be apparent to those skilled in the art.

### DETAILED DESCRIPTION OF THE INVENTION

In one aspect, the present invention provides a method for preventing CRF in a feline susceptible to developing CRF. The method comprises feeding the feline a composition comprising from about 28 to about 35% protein on a dry matter basis wherein the protein comprises at least about 75% vegetable protein. In some embodiments, the method comprises feeding the feline a composition that comprises from about 28 to about 31% protein wherein the protein comprises at least about 75% vegetable protein. In some such embodiments, the method comprises feeding the feline a composition that comprises from about 28 to about 30% protein. In other such embodiments, the method comprises feeding the feline a composition that comprises from about 29 to about 31% protein wherein the protein comprises at least about 75, at least about 80, at least about 85, at least about 90, or at least about 95% vegetable protein. In yet other such embodiments, the method comprises feeding the feline a composition that comprises from about 28 or about 28.5 to about 29, about 29.5, about 30, about 30.5, or about 31% protein wherein the protein comprises at least about 75, at least about 80, at least about 85, at least about 90, or at least about 95% vegetable protein. In some embodiments, the method comprises feeding the feline a composition selected from the compositions of the invention for preventing renal insufficiency. One skilled in the alt would understand that either a single composition of the invention can be fed to the feline or, alternatively, different such compositions can be fed to the feline for varying time intervals.

In other such embodiments, the method comprises feeding the feline a composition comprising from about 31 to about 35% protein wherein the protein comprises at least about 75% vegetable protein. In some such embodiments, the method comprises feeding the feline a composition comprising from about 32 to about 34% protein wherein the protein comprises at least about 75% vegetable protein. In other such embodiments, the method comprises feeding the feline a composition comprising from about 32 to about 35% protein wherein the protein comprises at least about 75% vegetable protein. In yet other such embodiments, the method comprises feeding the feline a composition comprising from about 33 to about 35% protein wherein the protein comprises at least about 75% vegetable protein. In further such embodiments, the method comprises feeding the feline a composition comprising from about 31, about 32, or about 33 to about 34 or about 35% protein wherein the protein comprises at least about 75, at least about 80, at least about 85, at least about 90, or at least about 95% vegetable protein. In yet further such embodiments, the method comprises feeding the feline a composition comprising from about 31 to about 32, about 33, about 34, or about 35% protein wherein the protein comprises at least about 75, at least about 80, at least about 85, at least about 90, or at least about 95% vegetable protein.

In some embodiments of the method for preventing CRF, the composition that is fed to the feline further comprises from about 0.45 to about 0.8% phosphorus on a dry matter basis. In some such embodiments, the composition that is fed to the feline comprises from about 0.45 to about 0.6% phosphorus, for example, from about 0.45 to about 0.55% or from about 0.45 to about 0.5% phosphorus. In other such embodiments, the composition fed to the feline comprises from about 0.6 to about 0.8% phosphorus, for example, from about 0.7 to about 0.8% or from about 0.6 to about 0.75% phosphorus.

In some embodiments of the method for preventing CRF, the composition that is fed to the feline further comprises from about 0.2 to about 0.35% sodium on a dry matter basis. In some such embodiments, the composition fed to the feline comprises from about 0.2 to about 0.25% sodium, and in other such embodiments, the composition fed to the feline comprises from about 0.2 to about 0.25% sodium.

In some embodiments of the method for preventing CRF, the composition that is fed to the feline further comprises from about 0.75 to about 1% potassium on a dry matter basis. In some such embodiments, the composition fed to the feline comprises from about 0.8 to about 1% potassium, and in other such embodiments, the composition comprises from about 0.75 to about 0.9% potassium.

In some embodiments of the method for preventing CRF, the composition fed to the feline further comprises two or more of phosphorus, sodium, and potassium in any combination of the amounts discussed above.

In another aspect, the present invention provides a method for treating CRF in a feline. The method comprises feeding the feline a composition comprising from about 28 to about 30% protein on a dry matter basis wherein the protein present in the composition comprises at least about 75% vegetable protein. In some embodiments, the method comprises feeding the feline a composition comprising from about 28 to about 29% protein wherein the protein comprises at least about 75% vegetable protein. In other such embodiments, the method comprises feeding the feline a composition comprising from about 29 to about 30% protein wherein the protein comprises at least about 75% vegetable protein. In some embodiments, the method comprises feeding the feline a composition comprising from about 28 or about 28.5 to about 29, about 29.5, or about 30% protein wherein the protein comprises at least about 75, at least about 80, at least about 85, at least about 90, or at least about 95% vegetable protein. In some embodiments, the method comprises feeding the feline a composition selected from the compositions of the invention for treating CRF. One skilled in the art would understand that either a single composition of the invention can be fed to the feline or, alternatively, different such compositions can be fed to the feline for varying time intervals.

In some embodiments of the method for treating CRF in a feline, the composition that is fed to the feline further comprises from about 0.45 to about 0.6% phosphorus on a dry matter basis. In some such embodiments, the composition fed to the feline comprises from about 0.45 to about 0.55% phosphorus. In other such embodiments, the composition fed to the feline comprises from about 0.45 to about 0.5% phosphorus.

In some embodiments of the method for treating CRF in a feline, the composition that is fed to the feline further comprises from about 0.2 to about 0.25% sodium on a dry matter basis.

In some embodiments of the method for treating CRF in a feline, the composition that is fed to the feline further comprises from about 0.75 to about 1% potassium on a dry matter basis. In some such embodiments, the composition that is fed to the feline comprises from about 0.7 to about 0.9% potassium. In other such embodiments, the composition fed to the feline comprises from about 0.75 to about 0.85% potassium.

In some embodiments of the method for treating CRF in a feline, the composition fed to the feline further comprises two or more of phosphorus, sodium, and potassium in any combination of the amounts discussed above.

In some embodiments, the methods of the invention for prevention and treatment of CRF further comprise administering to the feline an anti-CRF agent. An anti-CRF agent is a compound, a derivative thereof (e.g., a salt, solvate, or hydrate of the compound), or a composition comprising such compounds and/or derivatives that is used to prevent and/or treat CRF. Such an agent is administered in conjunction with feeding a composition of the invention. The term “in conjunction” means that an agent is administered to the feline either together with a composition of the present invention or separately from the composition at the same or different frequency via the same or different administration route and either at about the same time as the composition or periodically. “Administering” means that the agent is introduced in a suitable dosage form into the feline by a suitable administration route, for example, orally, topically, or parenterally. “About at the same time” generally means that an agent is administered when a composition of the invention is fed to the feline or within about 72 hours of feeding the composition to the feline. “Periodically” generally means that an agent is administered to a feline following a dosage schedule suitable for administering the agent while a composition of the invention is fed to the feline routinely as appropriate for that feline. Thus, the term “in conjunction” specifically includes situations when an agent is administered to a feline for a prescribed period of time while a composition of the invention is fed to the feline for a much longer period of time (e.g., for life). If more than one agent is administered, the dosage form and route of administration for each agent may vary. In addition, one composition of the invention may be substituted with another composition of the invention while a specific agent is administered to the feline.

Suitable anti-CRF agents include, for example, agents that reduce blood pressure or protein loss in urine, anabolic steroids, antibiotics, agents that treat anemia, and agents that control vomiting. Such agents can be administered, for example, in the form of salts derived from inorganic or organic acids. Depending on the particular compound, a salt of the compound may be advantageous due to one or more of the salt's physical properties, for example, enhanced pharmaceutical stability in differing temperatures and humidities, or a desirable solubility in water or oil. The salt preferably is a pharmaceutically-acceptable salt.

The total daily dose of an anti-CRF agent (administered in single or divided doses) is typically from about 0.001 to about 100 mg/kg, preferably from about 0.001 to about 30 mg/kg, and even more preferably from about 0.01 to about 10 mg/kg body weight. Dosage unit compositions can contain such amounts or submultiples thereof to make up the daily dose. In many instances, the administration of the anti-CRF agent will be repeated a plurality of times. Multiple doses per day typically may be used to increase the total daily dose. Factors affecting the preferred dosage regimen include, for example, the age, weight, and condition of the feline; the severity of the disease: the route of administration; pharmacological considerations (e.g., activity, efficacy, and pharmacokinetic and toxicology profiles of the particular anti-CRF agent); whether a drug delivery system is utilized; and whether the anti-CRF agent is administered as part of a drug combination. Thus, the dosage regimen actually employed can vary widely, and can differ from the dosage regimen set forth above.

In a further aspect, the invention provides a composition suitable for preventing CRF in a feline susceptible to developing renal insufficiency. The composition comprises from about 28 to about 35% protein on a dry matter basis wherein the protein comprises at least about 75% vegetable protein.

Since CRF is a terminal disease in felines, it is important to prevent a decrease in renal function in a feline that shows clinical signs of CRF. Such a feline is one which has not been diagnosed with CRF, but is at risk to develop CRF due to, for example, advanced age, genetic predisposition, or disease(s) or medication(s) that can damage the feline's kidney(s). Preventing CRF includes reducing the risk of, delaying the onset of, and/or keeping a feline from developing CRF. Thus, feeding a composition of the invention for preventing CRF can reduce a feline's risk of developing CRF. In addition, feeding such a composition can slow down the progression of loss of a feline's kidneys' built-in reserve, thereby delaying the onset of the later phases of CRF.

The invention's composition for preventing CRF is suitable for any feline which is susceptible to develop CRF. In some embodiments, the feline is a companion feline. A companion feline can be a feline kept as a pet. A companion feline can also be a feline from a widely domesticated species, for example, cats (Felis domesticus) regardless of whether or not it is kept as a pet. In some embodiments, the feline is an adult feline. An adult feline is a feline of any age after juvenile growth and development has been completed, including senior and geriatric felines. For example, an adult cat typically is one that is from about one year old through the remainder of its life. A senior feline is one of an age at which it is at a risk for suffering from an age-related disease regardless of whether or not the feline shows obvious physical or behavioral signs of aging. For example, a senior cat typically is a cat from about seven to about eleven years old. A geriatric feline is a feline showing signs of aging. For example, a geriatric cat typically is a cat of about twelve years of age and beyond.

The composition for preventing CRF in a feline comprises from about 28 to about 35% protein. Commercial adult cat foods, for example, typically comprise from about 35 to about 45% protein on a dry matter basis. Thus, in many embodiments, a composition of the invention for preventing CRF comprises less protein than most commercially available adult cat foods.

In some embodiments, the composition for preventing CRF comprises from about 28 to about 31% protein on a dry matter basis. In some such embodiments, the composition comprises from about 28 to about 30% protein; and in other such embodiments, the composition comprises from about 29 to about 31% protein. In yet other such embodiments, the composition comprises from about 28 or about 28.5 to about 29, about 29.5, about 30, about 30.5, or about 31% protein.

In other embodiments, the composition for preventing CRF comprises from about 31 to about 35% protein. In some such embodiments, the composition comprises from about 32 to about 34% protein. In other embodiments, the composition comprises from about 32 to about 35% protein. In other embodiments, the composition comprises from about 33 to about 35% protein. In other embodiments, the composition comprises from about 31, about 32, or about 33 to about 34 or about 35% protein. In further embodiments, the composition comprises from about 31 to about 32, about 33, about 34, or about 35% protein. Dietary protein restriction is beneficial for preventing and treating CRF. Excessive protein is catabolized to urea and other nitrogenous compounds normally excreted by the kidneys. Decreased renal function leads to accumulation of these compounds. Endogenous proteins will be degraded if amino acid intake is insufficient to maintain nitrogen balance. Thus, a composition of the invention for preventing and/or treating CRF can facilitate achieving nitrogen balance, and can help limit the accumulation of waste products by decreasing protein intake.

Because felines are carnivores, feline foods typically are formulated to comprise mostly animal and fish protein. To the contrary, the protein in a composition of the present invention for preventing CRF comprises at least about 75% vegetable protein (i.e., a composition of the invention for preventing CRF comprises from about 28 to about 35% protein wherein the protein present in the composition comprises at least about 75% vegetable protein). In some such embodiments, the composition comprises at least about 80% vegetable protein. In other embodiments, the composition comprises at least about 85% vegetable protein. In other such embodiments, the protein comprises at least about 90% vegetable protein. And in other such embodiments, the protein comprises at least about 95% vegetable protein (i.e., from at least about 95 tip to about 100% vegetable protein).

Thus, in some embodiments, the composition for preventing CRF comprises from about 28 to about 31% protein wherein the protein comprises at least about 75% vegetable protein. In some such embodiments, the composition comprises from about 28 to about 30% protein wherein the protein comprises at least about 75, at least about 80, at least about 85, at least about 90, or at least about 95% vegetable protein. In other such embodiments, the composition comprises from about 29 to about 31% protein wherein the protein comprises at least about 75, at least about 80, at least about 85, at least about 90, or at least about 95% vegetable protein. In yet other such embodiments, the composition comprises from about 28 or about 28.5 to about 29, about 29.5, about 30, about 30.5, or about 31% protein wherein the protein comprises at least about 75, at least about 80, at least about 85, at least about 90, or at least about 95% vegetable protein.

In other embodiments, the composition for preventing CRF comprises from about 31 to about 35% protein wherein the protein comprises at least about 75% vegetable protein. In some such embodiments, the composition comprises from about 31, about 32, or about 33 to about 34 or about 35% protein wherein the protein comprises at least about 75, at least about 80, at least about 85, at least about 90, or at least about 95% vegetable protein. In other embodiments, the composition comprises from about 31 to about 32, about 33, about 34, or about 35% protein, and the protein comprises at least about 75, at least about 80, at least about 85, at least about 90, or at least about 95% vegetable protein. In yet other embodiments, the composition comprises from about 28 or about 28.5 to about 29, about 29.5, or about 30% protein, and the protein comprises at least about 75, at least about 80, at least about 85, at least about 90, or at least about 95% vegetable protein.

Vegetarian diets generally contain higher amounts of fiber, magnesium folic acid, vitamin C, vitamin D, and carotenoids than non-vegetarian diets. Vegetarian diets generally contain less saturated fat, cholesterol, protein, sulfur-containing amino acids, calcium, and iodine than non-vegetarian diets. Phosphorus from vegetable protein is less available than phosphorus from animal protein. Thus, some characteristics of vegetarian diets (e.g. lower amount of protein, lower bioavailability of phosphorus, lower amount of sulfur-containing amino acids thereby reducing diet acidity) are consistent with the goals of the invention to prevent and/or treat CRF in felines. Plant ingredients suitable for preparing compositions of the invention for preventing and/or treating CRF include, for example, potato concentrate, soy concentrate, soy protein isolate, soybean meal, corn gluten meal, rice protein isolate, pea protein concentrate, wheat protein concentrate, and wheat protein isolate.

In some embodiments, the composition of the invention for preventing CRF in a feline further comprises from about 0.45 to about 0.8% phosphorus on a dry matter basis. In some such embodiments, the composition comprises about 0.45 to about 0.6% phosphorus, for example, from about 0.45 to about 0.55% or from about 0.45 to about 0.5% phosphorus. In other such embodiments, the composition comprises from about 0.6 to about 0.8% phosphorus, for example, from about 0.7 to about 0.8% or from about 0.6 to about 0.75% phosphorus. The term “phosphorus” means the phosphorus atom without reference to its molecular or ionic form. Secondary hyperparathyroidism and phosphorus retention have been incriminated as causes of CRF. Therefore, reduction of phosphorus in a feline's diet can be beneficial for preventing and/or treating CRF.

In some embodiments, the composition of the invention for preventing CRF in a feline further comprises from about 0.2 to about 0.35% sodium on a dry matter basis. In some such embodiments, the composition comprises from about 0.2 to about 0.25% sodium. In other such embodiments, the composition comprises from about 0.25 to about 0.3% sodium. The term “sodium” means the sodium atom without reference to its molecular or ionic form. When dietary sodium intake changes, fractional excretion of sodium must change as well to maintain sodium balance. Felines with CRF can only vary sodium excretion over a limited range, which narrows progressively as glomerular filtration rate declines. Thus, such felines cannot tolerate excessively high or excessively low dietary sodium intake. Feeding a feline a composition of the invention for preventing and/or treating CRF can help maintain optimal sodium balance.

In some embodiments, the composition of the invention for preventing CRF in a feline further comprises from about 0.75 to about 1% potassium on a dry matter basis. In some such embodiments, the composition comprises from about 0.8 to about 1% potassium. In other such embodiments, the composition comprises from about 0.75 to about 0.9% potassium. The term “potassium” means the potassium atom without reference to its molecular or ionic form. Normally, the majority of ingested potassium is excreted in the urine, with the remainder excreted in feces. The proportion of potassium excreted in feces increases in felines with CRF. Such felines typically also have disorders in potassium homeostasis. Feeding such felines a composition of the invention for preventing and/or treating CRF can help maintain optimal potassium balance.

In some embodiments, the composition of the invention for preventing CRF further comprises two or more of phosphorus, sodium, and potassium in any combination of the amounts discussed above.

In another aspect, the invention provides a composition suitable for treating CRF in a feline. The composition comprises from about 28 to about 30% protein on a dry matter basis wherein the protein comprises at least about 75% vegetable protein.

Treatment can help a feline with CRF to live a healthier, longer, and more active life. Treating CRF includes ameliorating and or suppressing CRF. One skilled in the art can diagnose CRF (as well as distinguish it from other diseases, for example, a kidney tumor, diabetes, and hyperthyroidism) by utilizing blood and urine tests as well as, for example, radiography, ultrasound, renal biopsy, and/or palpation. In addition to other symptoms, felines with CRF typically have an elevated serum creatinine and/or BUN A composition for treating CRF of the invention is suitable for any feline with CRF. Examples of felines that can be treated with a composition of the invention are discussed above in the context of the compositions of the invention for preventing CRF.

In some such embodiments, the composition comprises from about 28 to about 29% protein, and in other such embodiments, the composition comprises from about 29 to about 30% protein. In yet other such embodiments, the composition comprises from about 28 or about 28.5 to about 29, about 29.5, or about 30% protein. The protein in the composition for treating CRF comprises at least about 75% vegetable protein (i.e., a composition of the present invention for treating CRF in a feline comprises from about 28 to about 30% protein wherein the protein comprises at least about 75% vegetable protein). In some such embodiments, the composition comprises at least about 80% vegetable protein. In other such embodiments, the composition comprises at least about 85% vegetable protein. In other such embodiments, the protein comprises at least about 90% vegetable protein. In other such embodiments, the protein comprises at least about 95% vegetable protein (i.e., from at least about 95 up to about 100% vegetable protein). In some embodiments, the composition for treating CRF in a feline comprises from about 28 or about 28.5 to about 29, about 29.5, or about 30% protein wherein the protein comprises at least about 75, at least about 80, at least about 85, at least about 90, or at least about 95% vegetable protein.

In some embodiments, the composition for treating CRF further comprises from about 0.45 to about 0.6% phosphorus on a dry matter basis. In some such embodiments, the composition comprises from about 0.45 to about 0.55% phosphorus; and in other such embodiments from about 0.45 to about 0.5% phosphorus.

In some embodiments, the composition for treating CRF further-comprises from about 0.2 to about 0.25% sodium on a dry matter basis.

In some embodiments, the composition for treating CRF further comprises from about 0.75 to about 1% potassium on a dry matter basis. In some such embodiments, the composition comprises from about 0.7 to about 0.9% potassium; and in other such embodiments from about 0.75 to about 0.85% potassium.

In some embodiments, the composition for treating CRF further comprises two or more of phosphorus, sodium, and potassium in any combination of the amounts discussed above.

In some embodiments, the compositions of the invention for preventing and/or treating CRF comprise a food composition (i.e., the compositions comprise one or more food compositions). In some embodiments, the compositions meet or are below the AAFCO's minimum nutrient level requirements for reproduction or maintenance. In some embodiments, the food compositions comprise a dry food. In some embodiments, the food compositions comprise a semi-moist food. In some embodiments, the food compositions comprise a moist food. In some embodiments, the food compositions comprise a treat, snack, supplement, or partially or fully edible toy. In some embodiments, the compositions comprise a mixture of one or more foods.

In a further aspect, the invention provides a kit comprising a composition of the invention. The kit is suitable for preventing and/or treating CRF in a feline. In some embodiments, the kit further comprises an anti-CRF agent (i.e., one or more anti-CRF agents). In some embodiments, the kit further comprises instructions for one or more of (1) feeding the composition to a feline, (2) administering an anti-CRF agent to the feline in conjunction with feeding the feline the composition, (3) preventing CRF in a feline by feeding the feline the composition, (4) treating CRF in a feline by feeding the feline the composition, (5) preventing CRF in a feline by administering to the feline an anti-CRF agent in conjunction with feeding the feline the composition, and (6) treating CRF in a feline by administering to the feline an anti-CRF agent in conjunction with feeding the feline the composition.

In some embodiments, the kit comprises two or more ingredients that, when combined together and optionally with additional ingredients that are or are not a part of the kit, yield a composition of the present invention. If such additional ingredients are to be used, the kit provides instructions about these ingredients. In some embodiments, the kit further comprises an anti-CRF agent. In some embodiments, the kit further comprises instructions for one or more of (1) preparing a composition of the invention for preventing CRF in a feline by combining the ingredients, (2) preparing a composition of the invention for treating CRF in a feline by combining the ingredients, (3) feeding a feline a composition of the invention for preventing CRF, (4) feeding a feline a composition of the invention for treating CRF, (5) administering an anti-CRF agent to a feline in conjunction with feeding the feline a composition of the invention for preventing CRF, (6) administering an anti-CRF agent to a feline in conjunction with feeding the feline a composition of the invention for treating CRF, (7) preventing CRF in a feline by feeding the feline a composition of the invention for preventing CRF, (8) treating CRF in a feline by feeding the feline a composition of the invention for treating CRF, (9) preventing CRF in a feline by administering to the feline an anti-CRF agent in conjunction with feeding the feline a composition of the invention for preventing CRF, and (10) treating CRF in a feline by administering to the feline an anti-CRF agent in conjunction with feeding the feline a composition of the invention for treating CRF.

In some embodiments, the kit comprises in separate containers in a single package or in separate containers in a virtual package, as appropriate for the kit component, a composition of the invention or two or more ingredients, that, when combined together and optionally with additional ingredients that are or are not a part of the kit, yield a composition of the invention, and instructions for one or more of (1) preparing a composition of the invention for preventing CRF in a feline by combining the ingredients, (2) preparing a composition of the invention for treating CRF in a feline by combining the ingredients (3) feeding a feline a composition of the invention to prevent CRF, (4) feeding a feline a composition of the invention to treat CRF, (5) administering an anti-CRF agent to a feline in conjunction with feeding the feline a composition of the invention to prevent CRF, (6) administering an anti-CRF agent to a feline in conjunction with feeding the feline a composition of the invention to treat CRF, (7) preventing CRF in a feline by feeding the feline a composition of the invention for preventing CRF, (8) treating CRF in a feline by feeding the feline a composition of the invention for treating CRF, (9) preventing CRF in a feline by administering to the feline an anti-CRF agent in conjunction with feeding the feline a composition of the invention for preventing CRF, and (10) treating CRF in a feline by administering to the feline an anti-CRF agent in conjunction with feeding the feline a composition of the invention for treating CRF.

The term “single package” generally means that the components of a kit are physically associated in or with one or more containers and considered as a unit of manufacture, distribution, sale, or use. Containers include, for example, bags, boxes, bottles, shrink wrap packages, stapled or otherwise fixed components, and combinations thereof. A single package can be, for example, containers or individual food compositions physically associated such that they are considered a unit for manufacture, distribution, sale, or use. The term “virtual package” generally means that the components of a kit are associated by directions on one or more physical or virtual kit components instructing the user how to obtain additional components, e.g., in a bag containing one component and directions instructing the user to go to a website, contact a recorded message, view a visual message, or contact a caregiver to obtain instructions on how to use the kit. When the kit comprises a virtual package, the kit is limited to instructions in a virtual environment with one or more physical kit components.

In some embodiments, the kit is suitable for preventing chronic renal failure in a feline and comprises in separate containers in a single package or in separate containers in a virtual package, as appropriate, a composition that comprises from about 28 to about 35% protein on a dry matter basis wherein the protein comprises at least about 75% vegetable protein, or two or more ingredients that, when combined together and, optionally, with additional ingredients that are or are not a part of the kit, yield a composition that comprises from about 28 to about 35% protein wherein the protein comprises at least about 75% vegetable protein, and one or more of (1) an anti-chronic renal failure agent, (2) instructions for preparing the composition, (3) instructions for feeding the composition to a feline, (4) instructions for preventing chronic renal failure in a feline by feeding the feline the composition, and (5) instructions for preventing chronic renal failure in a feline by administering to the feline an anti-chronic renal failure agent in conjunction with feeding the feline the composition. In some such embodiments, the kit comprises a composition that comprises from about 31 to about 35% protein, or two or more ingredients that, when combined together and, optionally, with additional ingredients that are or are not a part of the kit, yield a composition that comprises from about 31 to about 35% protein.

In other embodiments, the kit is suitable for treating chronic renal failure in a feline and comprises in separate containers in a single package or in separate containers in a virtual package, as appropriate, a composition that comprises from about 28 to about 30% protein wherein the protein comprises at least about 75% vegetable protein, or two or more ingredients that, when combined together and, optionally, with additional ingredients that are or are not a part of the kit, yield a composition that comprises from about 28 to about 30% protein wherein the protein comprises at least about 75% vegetable protein, and one or more of (1) an anti-chronic renal failure agent, (2) instructions for preparing the composition, (3) instructions for feeding the composition to a feline, (4) instructions for treating chronic renal failure in a feline by feeding the feline the composition, and (5) instructions for treating chronic renal failure in a feline by administering to the feline an anti-chronic renal failure agent in conjunction with feeding the feline the composition.

In another aspect, the invention provides a means for communicating information about or instructions for one or more of (1) feeding a feline a composition of the invention to prevent CRF, (2) feeding a feline a composition of the invention to treat CRF, (3) administering an anti-CRF agent to a feline in conjunction with feeding the feline a composition of the invention to prevent CRF, (4) administering an anti-CRF agent to a feline in conjunction with feeding the feline a composition of the invention to treat CRF, (5) preventing CRF in a feline by feeding the feline a composition of the invention for preventing CRF, (6) treating CRF in a feline by feeding the feline a composition of the invention for treating CRF, (7) preventing CRF in a feline by administering to the feline an anti-CRF agent in conjunction with feeding the feline a composition of the invention for preventing CRF, (8) treating CRF in a feline by administering to the feline an anti-CRF agent in conjunction with feeding the feline a composition of the invention for treating CRF, (9) using a kit of the invention to prevent CRF, and (10) using a kit of the invention to treat CRF. The communication means comprise, for example, a document, digital storage media, optical storage media, audio presentation, or visual display containing the information or instructions. Preferably, the communication means is a displayed web site or a brochure, product label, package insert, advertisement, or visual display containing such information or instructions. Useful information or instructions include, for example, (1) information and instructions how to use a composition, method, or kit of the invention and (2) contact information for animal caregivers if they have a question about the invention and its uses.

In some embodiments, the present invention provides a means for communicating information about or instructions for one or more of (1) preventing chronic renal failure in a feline by feeding the feline a composition of the invention that comprises from about 28 to about 35% protein wherein the protein comprises at least about 75% vegetable protein, (2) preventing chronic renal failure in a feline by administering to the feline an anti-chronic renal failure agent in conjunction with feeding the feline a composition of the invention that comprises from about 28 to about 35% protein wherein the protein comprises at least about 75% vegetable protein, and (3) using a kit of the invention for preventing CRF in a feline that comprises a composition comprising from about 28 to about 35% protein wherein the protein comprises at least about 75% vegetable protein or two or more ingredients that, when combined together and, optionally, with additional ingredients, yield a composition that comprises from about 28 to about 35% protein wherein the protein comprises at least about 75% vegetable protein. The means comprise a document, digital storage media, audio presentation, or visual display containing the information or instructions.

In other embodiments, the present invention provides a means for communicating information about or instructions for one or more of (1) treating chronic renal failure in a feline by feeding the feline a composition of the invention that comprises from about 28 to about 30% protein on a dry matter basis wherein the protein comprises at least about 75% vegetable protein, (2) treating chronic renal failure in a feline by administering to the feline an anti-chronic renal failure agent in conjunction with feeding the feline a composition of the invention that comprises from about 28 to about 30% protein on a dry matter basis wherein the protein comprises at least about 75% vegetable protein, and (3) using a kit of the invention for treating CRF in a feline that comprises a composition comprising from about 28 to about 30% protein wherein the protein comprises at least about 75% vegetable protein or two or more ingredients that, when combined together and, optionally, with additional ingredients, yield a composition that comprises from about 28 to about 30% protein wherein the protein comprises at least about 75% vegetable protein. The communication means comprise a document, digital storage media, audio presentation, or visual display containing the information or instructions.

In another aspect, the invention provides for a use of a composition of the invention to prepare a medicament suitable for preventing feline CRF. As described herein, the composition generally comprises from about 28 to about 35% protein on a dry matter basis wherein the protein comprises at least about 75% vegetable protein. In some embodiments, the composition comprises from about 28 to about 31% protein wherein the protein present in the composition comprises at least about 75% vegetable protein. In some such embodiments, the composition comprises from about 28 to about 30% protein wherein the protein comprises at least about 75, at least about 80, at least about 85, at least about 90, or at least about 95% vegetable protein. In other such embodiments, the composition comprises from about 29 to about 31% protein wherein the protein comprises at least about 75, at least about 80, at least about 85, at least about 90, or at least about 95% vegetable protein. In yet other such embodiments, the composition comprises from about 28 or about 28.5 to about 29, about 29.5, about 30, about 30.5, or about 31% protein Wherein the protein comprises at least about 75, at least about 80, at least about 85, at least about 90, or at least about 95% vegetable protein. In other embodiments, the composition comprises from about 31 to about 35% protein wherein the protein comprises at least about 75% vegetable protein. In some such embodiments, the composition comprises from about 31, about 32, or about 33 to about 34 or about 35% protein wherein the protein comprises at least about 75, at least about 80, at least about 85, at least about 90, or at least about 95% vegetable protein. In other such embodiments, the composition comprises from about 31 to about 32, about 33, about 34, or about 35% protein wherein the protein comprises at least about 75, at least about 80, at least about 85, at least about 90, or at least about 95% vegetable protein. In some embodiments, the composition further comprises one or more of sodium, phosphorus, and potassium in the amounts discussed above. And in some embodiments, the composition comprises a food composition.

In another aspect, the invention provides for a use of a composition of the invention to prepare a medicament suitable for treating feline CRF. As described herein, the composition generally comprises from about 28 to about 30% protein on a dry matter basis wherein the protein comprises at least about 75% vegetable protein. In some embodiments, the composition comprises from about 28 or about 28.5 to about 29, about 29.5, or about 30% protein wherein the protein comprises at least about 75, at least about 80, at least about 85, at least about 90, or at least about 95% vegetable protein. In some embodiments, the composition further comprises one or more of phosphorus, sodium, and potassium in the amounts discussed above. And in some embodiments, the composition comprises a food composition.

Generally, medicaments are prepared by admixing a compound or composition with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to a feline.

In another aspect, the invention provides methods for preparing compositions of the invention for preventing and/or treating CRF. Such composition can be prepared, for example, by mixing various ingredients (including food compositions) that, when combined, yield a composition of the invention. Compositions of the invention can also be prepared by the methods discussed in, for example, Small Animal Nutrition, pages 127-146 (2000). As discussed above, plant ingredients suitable for preparing compositions of the invention include, for example, potato concentrate, soy concentrate, soybean meal, soy protein isolate, corn gluten meal, rice protein isolate, pea protein concentrate, wheat protein concentrate, and wheat protein isolate.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a, “an,” and “the” include plural reference unless the context clearly dictates otherwise. Similarly, the words comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and other references mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

### EXAMPLES

The invention can be further illustrated by the following examples, although it will be understood that the examples are included merely for purposes of illustration and are not intended to limit the scope of the invention unless otherwise specifically indicated.

### Example 1

Example 1 illustrates the effect of the compositions of the invention on preventing CRF in healthy cats.

Food A was formulated as a dry cat food meeting the AAFCO's minimum nutrient level requirements for maintenance for cats. It contained soybean meal, corn gluten meal, poultry meal, and pork meat protein isolate as protein ingredients. Food A contained approximately 34% total protein on a dry matter basis: 97% of the protein was from vegetable or grain.

Food B1 was formulated as a dry cat food meeting AAFCO's minimum nutrient level requirements for maintenance for cats. It contained soybean meal, corn gluten meal, poultry meal, and pork meat protein isolate as protein ingredients. Food B1 contained approximately 34% total protein on a dry matter basis; 77% of the protein was from vegetable.

Food B2 was formulated as a wet cat food meeting AAFCO's minimum nutrient level requirements for maintenance for cats. It contained soybean meal, corn gluten meal, pork lungs, chicken, and pork liver as protein ingredients. Food B2 contained approximately 34% total protein on a dry matter basis: 32% of the protein was from vegetable.

Food C was formulated as a dry cat food meeting AAFCO's minimum nutrient level requirements for maintenance for cats. It containing corn gluten meal, poultry meal, and pork meat protein isolate as protein ingredients. Food C contained approximately 34% total protein on a dry matter basis; 85% of the protein was from vegetable.

Eight healthy cats were fed food A for 26 weeks. Another eight healthy cats were fed food C for 26 weeks. Six healthy cats were fed food B1 for 26 weeks. And another six healthy cats were fed food B2 for 26 weeks. The serum creatinine levels of all cats were measured at the beginning and at the completion of the study (i.e., at day 0 and at 26 weeks, respectively). The results are presented in Table 1.

TABLE 1
Change in Serum Creatinine Levels

| | | % Protein | | | |
|---|---|---|---|---|---|
| | Total | from | Creatinine | Creatinine | Creatinine |
| | protein | Vegetable | at day 0 | at week 26 | change |
| Food | (% DMB) | (% DMB) | (mg/dL) | (mg/dL) | (mg/dL) |
| Food A | 34 | 97.0 | 1.53 | 1.14 | −0.39 |
| Food B1 | 34 | 76.5 | 1.28 | 1.22 | −0.06 |
| Food B2 | 34 | 32.0 | 1.48 | 1.39 | −0.09 |
| Food C | 34 | 85.0 | 1.46 | 1.10 | −0.36 |

All cats had normal serum creatinine level at the beginning of the study. Serum creatinine levels of approximately 0.8 to approximately 1.8 mg/dL are considered normal. The cats that were fed food B1 had the lowest initial serum creatinine level, A significant reduction in serum creatinine level was observed in the cats fed foods A and C (i.e., the foods with the highest vegetable protein content), particularly when compared to the cats fed food B2 (i.e., the food with the lowest vegetable protein content). While no reduction in serum creatinine level was observed in the cats fed foods B1 (i.e., a food with high vegetable content) compared to the cats fed food B2 was observed, the initial serum creatinine level in the cats fed food B1 was lower than the serum creatinine level for fed food A, B2, or C. Thus, the fact that the cats fed food B1 had the lowest creatinine level at the beginning of the study explains the lower decrease in serum creatinine level in those cats.

### Example 2

Example 2 illustrates the effect of the compositions of the invention on treating CRF in cats with renal sufficiency and age matched normal cats.

Food D was formulated as a dry cat food meeting the AAFCO's minimum nutrient level requirements for maintenance for cats. It contained approximately 28% total protein on a dry matter basis; 79% of the protein was from vegetable origin.

Six cats diagnosed with renal insufficiency and 6 healthy cats were fed food D for 26 weeks. The serum creatinine and serum phosphorus levels of all cats were measured at the beginning of the study (i.e., at day 0) and at 13 and 26 weeks. The results are presented in Tables 2 and 3.

TABLE 2
Change in Serum Creatinine Levels

| | Creatinine | Creatinine | Creatinine |
|---|---|---|---|
| | at day 0 (mg/dL) | at week 13 (mg/dL) | change (mg/dL) |
| CRF cats | 2.44 | 2.15 | −0.29 |
| Healthy cats | 1.25 | 1.03 | −0.22 |

TABLE 3
Change in Serum Phosphorus Levels

| | Phosphorus | Phosphorus | Phosphorus |
|---|---|---|---|
| | at day 0 (mg/dL) | at week 26 (mg/dL) | change (mg/dL) |
| CRF cats | 4.67 | 4.46 | −0.21 |
| Healthy cats | 4.98 | 4.64 | −0.34 |

Feeding food D reduced serum creatinine levels in both the healthy cats and the cats with CRF. In addition, feeding food D also resulted in reduction in serum phosphorus level in both the healthy cats and the cats with CRF (phosphorus levels of approximately 3 to approximately 5.6 mg/dL are considered normal).

Unless otherwise stated, all percentages provided herein are weight percentages on a dry matter basis. The term “dry matter basis” (DMB) means that an ingredient's concentration in a composition is measured after removing the moisture from the composition.

In the specification, there have been disclosed typical preferred embodiments of the invention and, although specific terms are employed, they are used in a generic and descriptive sense only and not for purposes of limitation, the scope of the invention being set forth in the claims. Obviously many modifications and variations of the invention are possible in light of the above teachings. It is therefore to be understood that within the scope of the appended claims the invention may be practiced otherwise than as specifically described.

1. A method for preventing chronic kidney disease in a feline comprising feeding the feline a composition comprising from about 28 to about 38% protein on a dry matter basis, wherein the protein comprises at least about 75% vegetable protein.

2. The method of claim 1 wherein the composition comprises from about 31 to about 35% protein.

3. The method of claim 1 wherein the composition further comprises from about 0.6 to about 0.8% phosphorus on a dry matter basis.

4. The method of any one of claims 1 wherein the composition further comprises from about 0.25 to about 0.35% sodium on a dry matter basis.

5. The method of any one of claims 1 wherein the composition further comprises from about 0.75 to about 1% potassium on a dry matter basis.

6. The method of any one of claims 1 wherein the feline is a cat.

7. The method of any one of claims 1 wherein the composition is a food composition.

8. A method for treating chronic kidney disease in a feline comprising feeding the feline a composition that comprises from about 26 to about 30% protein on a dry matter basis wherein the protein comprises at least about 75% vegetable protein.

9. The method of claim 8 wherein the composition further comprises from about 0.20 to about 0.50% phosphorus.

10. The method of claim 8 wherein the composition further comprises from about 0.23 to about 0.30% sodium on a dry matter basis.

11. The method of any one of claims 8 wherein the composition further comprises from about 0.75 to about 1% potassium on a dry matter basis.

12. The method of any one of claims 8 wherein the feline is a cat.

13. The method of any one of claims 8 wherein the composition is a food composition.

14. The method of claim 8 wherein the method further comprises administering to the feline an anti-chronic kidney disease agent.

15. A composition comprising from about 28 to about 38% protein on a dry matter basis wherein the protein comprises at least about 75% vegetable protein.

16. The composition of claim 15 wherein the composition comprises from about 31 to about 35% protein.

17. The composition of claim 15 wherein the composition further comprises from about 0.6 to about 0.8% phosphorus on a dry matter basis.

18. The composition of any one of claims 15 wherein the composition further comprises from about 0.25 to about 0.35% sodium on a dry matter basis.

19. The composition of any one of claims 15 wherein the composition further comprises from about 0.75 to about 1% potassium on a dry matter basis.

20. The composition of any one of claims 15 wherein the feline is a cat.

21. The composition of any one of claims 15 wherein the composition is a food composition.

22. The composition of any one of claims 15 suitable for preventing chronic kidney disease in a feline.

23. A composition comprising from about 26 to about 30% protein on a dry matter basis wherein the protein comprises at least about 75% vegetable protein.

24. The composition of claim 23 wherein the composition further comprises from about 0.20 to about 0.50% phosphorus on a dry matter basis.

25. The composition of claim 23 wherein the composition further comprises from about 0.23 to about 0.30% sodium on a dry matter basis.

26. The composition of any one of claims 23 wherein the composition further comprises from about 0.75 to about 1% potassium on a dry matter basis.

27. The composition of any one of claims 23 wherein the feline is a cat.

28. The composition of any one of claims 23 wherein the composition is a food composition.

29. The composition of any one of claims 23 suitable for treating chronic kidney disease in a feline.

30. A kit suitable for preventing chronic kidney disease in a feline comprising in separate containers in a single package or in separate containers in a virtual package, as appropriate for the kit component, a composition that comprises from about 28 to about 38%, or from about 31 to about 35% protein on a dry matter basis wherein the protein comprises at least about 75% vegetable protein, or two or more ingredients that, when combined together and, optionally, with additional ingredients that are or are not a part of the kit, yield a composition that comprises from about 28 to about 38%, or from about 31 to about 35% protein on a dry matter basis wherein the protein comprises at least about 75% vegetable protein, and one or more of (1) an anti-chronic kidney disease agent, (2) instructions for preparing the composition, (3) instructions for feeding the composition to a feline, (4) instructions for preventing chronic kidney disease in a feline by feeding the feline the composition, and (5) instructions for preventing chronic kidney disease in a feline by administering to the feline an anti-chronic kidney disease agent in conjunction with feeding the feline the composition.

31. The kit of claim 30 wherein the kit comprises a composition that comprises from about 31 to about 35% protein, or two or more ingredients that, when combined together and, optionally, with additional ingredients that are or are not a part of the kit, yield a composition that comprises from about 31 to about 35% protein.

32. A kit suitable for treating chronic kidney disease in a feline wherein the kit comprises in separate containers in a single package or in separate containers in a virtual package, as appropriate, a composition that comprises from about 26 to about 30% protein on a dry matter basis wherein the protein comprises at least about 75% vegetable protein, or two or more ingredients that, when combined together and, optionally, with additional ingredients that are or are not a part of the kit, yield a composition that comprises from about 26 to about 30% protein on a dry matter basis wherein the protein comprises at least about 75% vegetable protein, and one or more of (1) an anti-chronic kidney disease agent, (2) instructions for preparing the composition, (3) instructions for feeding the composition to a feline, (4) instructions for treating chronic kidney disease in a feline by feeding the feline the composition, and (5) instructions for treating chronic kidney disease in a feline by administering to the feline an anti-chronic kidney disease agent in conjunction with feeding the feline the composition.

33. A means for communicating information about or instructions for one or more of (1) preventing chronic kidney disease in a feline by feeding the feline a composition that comprises from about 28 to about 38% protein on a dry matter basis wherein the protein comprises at least about 75% vegetable protein, (2) preventing chronic kidney disease in a feline by administering to the feline and anti-chronic kidney disease agent in conjunction with feeding the feline a composition that comprises from about 28 to about 38% protein on a dry matter basis wherein the protein comprises at least about 75% vegetable protein, and (3) using a kit of claim 30 the means comprising a document, digital storage media, audio presentation, or visual display containing the information or instructions.

34. A means for communicating information about or instructions for one or more of (1) treating chronic kidney disease in a feline by feeding the feline a composition that comprises from about 26 to about 30% protein on a dry matter basis wherein the protein comprises at least about 75% vegetable protein, (2) treating chronic kidney disease in a feline by administering to the feline and anti-chronic kidney disease agent in conjunction with feeding the feline a composition that comprises from about 26 to about 30% protein on a dry matter basis wherein the protein comprises at least about 75% vegetable protein, and (3) using a kit of claim 32 the means comprising a document, digital storage media, audio presentation, or visual display containing the information or instructions.

35. A use of a composition that comprises from about 28 to about 38% protein on a dry matter basis wherein the protein comprises at least about 75% vegetable protein to prepare a medicament suitable for preventing chronic kidney disease in a feline.

36. A use of a composition that comprises from about 26 to about 30% protein on a dry matter basis wherein the protein comprises at least about 75% vegetable protein to prepare a medicament suitable for treating chronic kidney disease in a feline.

37. A method for lowering the creatinine serum level in an animal comprising administering to said animal a composition in accordance with claims 15.

38. A method for lowering the creatinine serum level in an animal comprising administering to said animal a composition in accordance with claim 23.
